(19) United States
(12) Patent Application Publication
(10) Pub. No.: US 2007/0093486 A1
(43) Pub. Date: Apr. 26, 2007

(54) Enzyme-catalyzed dynamic kinetic resolution process for preparing (+)-(2s,3s)-2-(3-chlorophenyl)-3,5,5-trimethyl-2-morpholinoland salts and solvates thereof

(75) Inventor: John Joseph Partridge, Durham, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Smith Kline beecham Corporation ,Corporation

(21) Appl. No.: 10/577,410

(22) PCT Filed: Oct. 21, 2004
(86) PCT No.: PCT/US04/34755

§ 371 Date: Apr. 26, 2006

Related U.S. Application Data
(60) Provisional application No. 60/514,694, filed on Oct. 27, 2003.

(51) Int. Cl. A61K 31/5375; C12P 17/00

(52) U.S. Cl. 514/237.5; 435/117

(57) ABSTRACT

A process for preparing (+)-(2S,3S)-2-(3-chlorophenyl)-3,5,5-trimethyl-2-morpholinol, pharmaceutically acceptable salts and solvates thereof such as the (+)-(2S,3S)-2-(3-chlorophenyl)-3,5,5-trimethyl-2-morpholinol hydrochloride salt via enzyme-catalyzed dynamic kinetic resolutions is provided.

### BACKGROUND OF THE INVENTION

1. Field of the Invention

The present invention relates to a process for making (+)-(2S,3S)-2-(3-chlorophenyl)-3,5,5-trimethyl-2-morpholinol, pharmaceutically acceptable salts, and pharmaceutically acceptable solvates thereof, such as the (+)-(2S,3S)-2-(3-chlorophenyl)-3,5,5-trimethyl-2-morpholinol hydrochloride salt by an enzyme-catalyzed dynamic kinetic resolution of the racemate (+/−)-(2R*,3R*)-2-(3-chlorophenyl)-3,5,5-trimethyl-2-morpholinol.

2. Description of the Prior Art

(+)-(2S,3S)-2-(3-chlorophenyl)-3,5,5-trimethyl-2-morpholinol, pharmaceutically acceptable salts thereof, and pharmaceutically acceptable solvates thereof, and pharmaceutical compositions comprising the same are used in treating numerous diseases or disorders such as depression, attention deficit hyperactivity disorder (ADHD), obesity, migraine, pain, sexual dysfunction, Parkinson's disease, Alzheimer's disease, seasonal affective disorder (SAD), addiction to alcohol, addiction to cocaine, or addiction to nicotine-containing (especially tobacco) products.

Several literature references describe the preparation of either the (+)-(2S,3S) or (−)-(2R,3R)-enantiomers from (+/−)-(2R*,3R*)-2-(3-chlorophenyl)-3,5,5-trimethyl-2-morpholinol. For instance, reference is made to U.S. Pat. No. 6,342,496 B1, issued to Jerussi et al. on Jan. 29, 2002, U.S. Pat. No. 6,337,328 B1, issued to Fang et al. on Jan. 8, 2002, U.S. Published Applications 2002/0052340 A1, and 2002/0052341 A1, as well as WO 01/62257 A2. Reference is also made to pending U.S. application Ser. No. 10/147,588; to U.S. Pat. No. 6,274,579; to U.S. Pat. No. 6,391,875; and to U.S. Pat. No. 6,734,213.

U.S. Pat. No. 6,337,328 B1, U.S. Published Application 2002/0052341 A1, WO 01/62257 A2, and U.S. application Ser. No. 10/147,588 refer to a chiral acid resolution method for preparing (+)-(2S,3S)-2-(3-chlorophenyl)-3,5,5-trimethyl-2-morpholinol from the racemate (+/−)-(2R*,3R*)-2-(3-chlorophenyl)-3,5,5-trimethyl-2-morpholinol. However, the method described in each of these references differs from the present invention in both procedure and result. These references relate to chemical resolutions of the racemate, while the present invention involves the enzyme-catalyzed dynamic kinetic resolution of the racemate. In the simple chemical resolution of the racemate, these references must isolate the desired diastereomeric morpholinol from a mixture of diastereomeric salts. The maximum yield of the desired diastereomer can therefore be at most about 50%.

In general, most chemical resolutions of a racemic material, such as Fang et al and Jerussi et al, produce the desired enantiomer or mirror image diastereoisomer in a maximum theoretical yield of 50%. Generally, the undesired enantiomer or mirror image diastereoisomer is discarded as waste in the mother liquor.

There are a few instances in which a maximum theoretical yield of 100% of a particular specific enantiomer can be obtained by a chiral enzymatic reaction on a pro-chiral substrate. This process is sometimes termed “an enzymatic hydrolytic desymmetrization”. Such a chiral enzymatic reaction on a pro-chiral substrate for five closely related compounds is set forth in “Enantioselective Hydrolysis of cis-3,5-Diacetoxycyclopentene: 1R,4S-(+)-4-Hydroxy-2-cyclopentenyl Acetate”, Deardorff, D. R., Windham, C. Q. and Craney, C. L., Org. Synth. Coll. Vol. IX, 1998, 487-493.

Likewise, there are few instances in which a dynamic kinetic resolution can be employed to give a maximum theoretical yield of 100% of a desired specified enantiomer, via equilibration of the enantiomers during the resolution. An example of this rare type of chemical dynamic kinetic resolution can be found in Reider, P. J., Davis, P., Hughes, D. L. and Grabowski, E. J., J. Org. Chem., 1987, 52, 955. An example of a rare alpha-substituted ketone reductive dynamic kinetic resolution leading largely to a single diastereoisomer is described in Yamada, S., Mori, Y., Morimatsu, K., Ishizu, Y., Ozaki, Y., Yoshioka, R., Nakatani, T., and Seko, H., J. Org. Chem., 1996, 61, 8586. In general, true enzyme-catalyzed diastereoisomeric dynamic kinetic resolutions are extremely rare for the preparation of a single, pure diastereoisomer (a compound containing two chiral centers), since both chiral centers must be capable of equilibration. In this special case of enzyme-catalyzed diastereoisomer dynamic kinetic resolutions, only one of four possible chiral diastereoisomers is formed.

### SUMMARY OF THE INVENTION

There exists a need for a reaction to produce (+)-(2S,3S)-2-(3-chlorophenyl)-3,5,5-trimethyl-2-morpholinol from its two chiral center-containing racemate in a greater than about 50% yield as is typical in a simple chemical resolution isolation process. There especially exists the need to produce this compound in a yield approximating 100%, that is, greater than about 60% yield, preferably greater than about 75% yield. Therefore, according to the present invention, there is provided an enzyme-catalyzed dynamic kinetic resolution method of preparing the compound (+)-(2S,3S)-2-(3-chlorophenyl)-3,5,5-trimethyl-2-morpholinol and/or desired salt and solvate forms from the corresponding racemate, which racemate will be referred to herein as (+/−)-(2R*,3R*)-2-(3-chlorophenyl)-3,5,5-trimethyl-2-morpholinol. There also exists the need to produce (+)-(2S,3S)-2-(3-chlorophenyl)-3,5,5-trimethyl-2-morpholinol and/or desired salt and solvate forms in higher yield in a commercial process that is inexpensive and environmentally feasible.

It is believed that when the present invention is compared with prior methods of isolation (e.g., a simple isolation or resolution), it will be apparent that according to the present invention, there will be a much higher yield (greater than about 50% yield and generally greater than about 80% yield). Further, probably there will be no need to isolate a chiral acid salt of (+)-(2S,3S)-2-(3-chlorophenyl)-3,5,5-trimethyl-2-morpholinol from the mixture of diastereomeric salts. Additionally, it is believed that there will be little or no mother liquor left over for disposal as waste.

The present invention achieves one or more of these desirable results by performing an enzyme-catalyzed dynamic kinetic resolution in a range of acidic to mildly basic pH's, namely about pH 1 to about pH 8. As can be seen in the schematic diagram, this allows the undesired 2R,3R-morpholinol (co-equal structures 3 and 4) is allowed to enzymatically “unravel” to the undesired 2R-hydroxybupropion (intermediate D), then to be enzymatically racemized or equilibrated (intermediate D→intermediate C→intermediate B)→intermediate A) with the desired 2S-hydroxybupropion (intermediate A), which then enzymatically “ravels” back up to the desired 2S,3S-morpholinol (co-equal structures 1 and 2) which crystallizes out as a solid free base, or solid acid salt, or mixtures thereof. This method will produce a desired free base or desired acid salt or mixtures thereof in about 70 to about 100% yield, preferably about 80 to about 100% yield, with little or no mother liquor left over. This is beneficial to the environment and/or eliminates further processing of the mother liquor before disposing of it. Additionally, a chiral acid resolving agent is not needed in this process. The absence of a chiral acid resolving agent further reduces manufacturing costs.

In short, there is provided a process for preparing (+)-(2S,3S)-2-(3-chlorophenyl)-3,5,5-trimethyl-2-morpholinol in its free base, it salt form, or both, which process comprises an enzyme-catalyzed dynamic kinetic resolution by equilibrating the two chiral centers of (+/−)-(2R*,3R*)-2-(3-chlorophenyl)-3,5,5-trimethyl-2-morpholinol.

To summarize, the present invention provides a process for making (+)-(2S,3S)-2-(3-chlorophenyl)-3,5,5-trimethyl-2-morpholinol which process comprises:

(1) dissolving (+/−)-(2R*,3R*)-2-(3-chlorophenyl)-3,5,5-trimethyl-2-morpholinol in a solvent and water and adjusting the pH from about pH 1 to about pH 8;

(2) adding a catalytic amount of an esterase enzyme or a lipase enzyme (preferably slowly) optionally with stirring;

(3) adding seed crystals selected from the group consisting of (i) (+)-(2S,3S)-2-(3-chlorophenyl 3,5,5-trimethyl-2-morpholinol free base, (ii) a salt of (+)-(2S,3S)-2-(3-chlorophenyl)-3,5,5-trimethyl-2-morpholinol, and (iii) a mixture of said free base and said salt of (+)-(2S,3S)-2-(3-chlorophenyl)-3,5,5-trimethyl-2-morpholinol, while maintaining the pH between about pH 1 and about pH 8 and the reaction temperature between about 10° C. and about 50° C.;

(4) quenching the reaction with an organic solvent and a base;

(5) removing said esterase enzyme or said lipase enzyme from the reaction; and

(6) isolating (+)-(2S,3S)-2-(3-chlorophenyl)-3,5,5-trimethyl-2-morpholinol free base.

In a preferred embodiment, the present invention provides a process for making (+)-(2S,3S)-2-(3-chlorophenyl)-3,5,5-trimethyl-2-morpholinol in which the free base and/or its salt form is further purified and/or “polished”, thereby making it more acceptable for animal, especially human consumption. This preferred process comprises:

(1) dissolving (+/−)-(2R*,3R*)-2-(3-chlorophenyl)-3,5,5-trimethyl-2-morpholinol in a solvent and water and adjusting the pH to about pH 1 to about pH 8;

(2) adding a catalytic amount of an esterase enzyme or a lipase enzyme (preferably slowly), optionally with stirring;

(3) adding seed crystal selected from the group consisting of (i) (+)-(2S,3S)-2-(3-chlorophenyl)-3,5,5-trimethyl-2-morpholinol free base, (ii) a salt of (+)-(2S,3S)-2-(3-chlorophenyl)-3,5,5-trimethyl-2-morpholinol, and (iii) a mixture of said free base and said salt of (+)-(2S,3S)-2-(3-chlorophenyl)-3,5,5-trimethyl-2-morpholinol, while maintaining the pH between about pH 1 and about pH 8 and the reaction temperature between about 10° C. and about 50° C.;

(4) quenching the reaction with an organic solvent and a base;

(5) removing said esterase enzyme or said lipase enzyme from the reaction;

(6) isolating (+)-(2S,3S)-2-(3-chlorophenyl)-3,5,5-trimethyl-2-morpholinol free base;

(7) converting the (+)-(2S,3S)-2-(3-chlorophenyl)-3,5,5-trimethyl-2-morpholinol free base into a (+)-(2S,3S)-2-(3-chlorophenyl)-3,5,5-trimethyl-2-morpholinol salt; and

(8) recrystallizing said salt to produce a purer form of the (+)-(2S,3S)-2-(3-chlorophenyl)-3,5,5-trimethyl-2-morpholinol salt.

The schematic diagram set forth below illustrates the present enzyme-catalyzed or enzymatic dynamic kinetic resolution method for morpholinols:

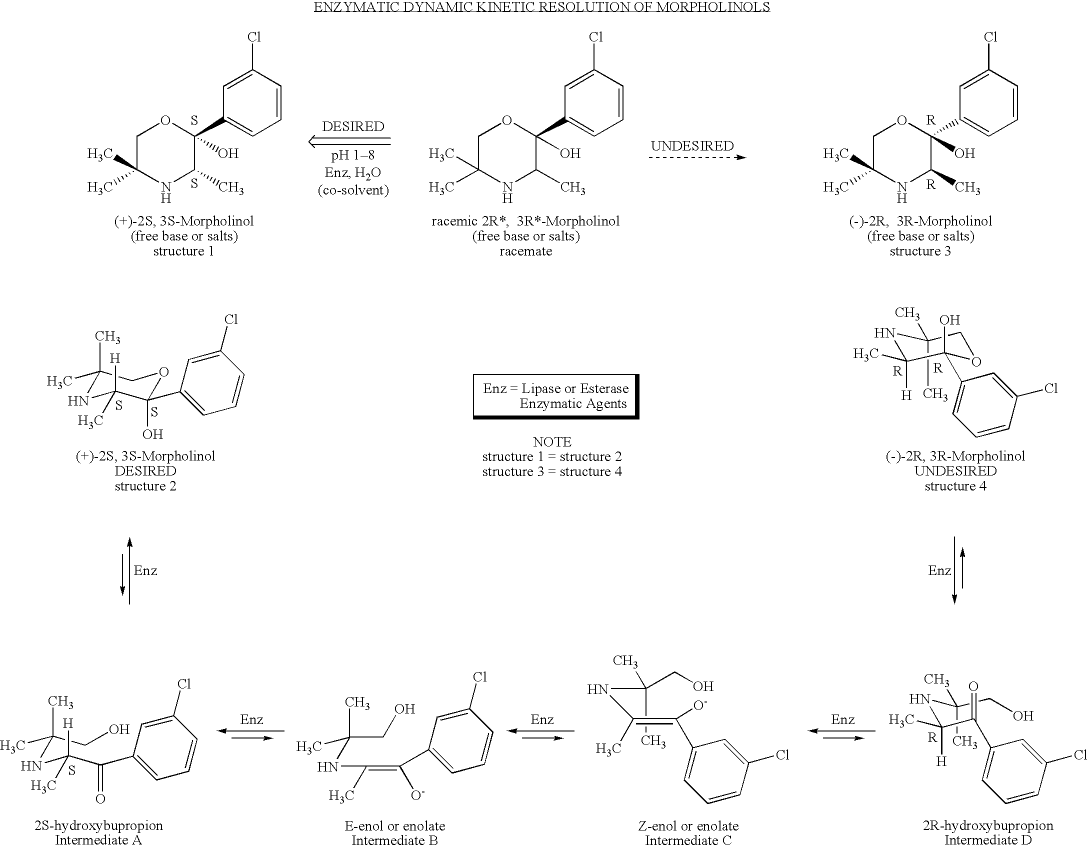

In a further embodiment of the invention there is provided a pharmaceutical composition comprising an active ingredient of (+)-(2S,3S)-2-(3-chlorophenyl)-3,5,5-trimethyl-2-morpholinol, a pharmaceutically acceptable salt thereof, or pharmaceutically acceptable solvate thereof prepared in accordance with the process described herein together with at least one pharmaceutically acceptable excipient.

In still another embodiment of the invention there is provided a method of treatment comprising the administration (preferably oral) to a mammal of an active ingredient comprising (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with the process described herein together with at least one pharmaceutically acceptable excipient.

In another embodiment there is provided the use of (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with process described herein in the manufacture of a medicament. Such medicament can be used for the treatment of depression (major and bipolar), attention deficit hyperactivity disorder (ADHD), anxiety, obesity, migraine, pain, sexual dysfunction in both men and women, Parkinson's disease, Alzheimer's disease, seasonal affective disorder (SAD), addiction to alcohol, addiction to cocaine, or addiction to nicotine-containing products (e.g., tobacco).

### DETAILED DESCRIPTION OF THE INVENTION

1. Introduction

The present invention provides a method for making (+)-(2S,3S)-2-(3-chlorophenyl)-3,5,5-trimethyl-2-morpholinol, a single diastereoisomer from a two-chiral center racemate. The process is an unusual example of an “enzyme-catalyzed” or “enzyme-induced” asymmetric transformation, also termed a “second-order asymmetric transformation”, but, importantly with two chiral centers equilibrating. (For one chiral center equilibrating asymmetric transformations see “Crystallization-Induced Asymmetric Transformations” by Jacques, J., Collet, A. and Wilen, S. H., in Enantiomers, Racemates and Resolutions (Krieger Publishing Company, Malabar, Fla.), 1991, Chapter 6, pp. 369-377. These processes are also referred to as a dynamic kinetic resolutions as disclosed in “Enantioselective Synthesis: The Optimum Solution”, Partridge, J. J. and Bray, B. L. in Process Chemistry in the Pharmaceutical Industry, (Gadamasetti, K. G., Ed.) Marcel Dekker, New York, N.Y., 1999, pp. 314-315.

In the process of the invention the following steps are performed:

(1) dissolving (+/−)-(2R*,3R*)-2-(3-chlorophenyl)-3,5,5-trimethyl-2-morpholinol in a solvent and water and adjusting the pH to about pH 1 to about pH 8;

(2) adding a catalytic amount of an esterase enzyme or a lipase enzyme optionally with stirring;

(3) adding seed crystals selected from the group consisting of (i) (+)-(2S,3S)-2-(3-chlorophenyl)-3,5,5-trimethyl-2-morpholinol free base, (ii) a salt of (+)-(2S,3S)-2-(3-chlorophenyl)-3,5,5-trimethyl-2-morpholinol, and (iii) a mixture of said free base and said salt of (+)-(2S,3S)-2-(3-chlorophenyl)-3,5,5-trimethyl-2-morpholinol, while maintaining the pH between about 1 and about 8 and the reaction temperature between about 10 C.° and about 50° C.;

(4) quenching the reaction with an organic solvent and a base;

(5) removing said esterase enzyme or said lipase enzyme from the reaction; and

(6) isolating (+)-(2S,3S)-2-(3-chlorophenyl)-3,5,5-trimethyl-2-morpholinol free base.

The following additional steps are performed in a preferred embodiment:

(7) converting the (+)-(2S,3S)-2-(3-chlorophenyl)-3,5,5-trimethyl-2-morpholinol free base into a precipitated form (either amorphous and/or crystalline) of (+)-(2S,3S)-2-(3-chlorophenyl)-3,5,5-trimethyl-2-morpholinol salt; and

(8) recrystallizing said salt to produce a purer form of a (+)-(2S,3S)-2-(3-chlorophenyl)-3,5,5-trimethyl-2-morpholinol salt.

The objective of the above-described process is to produce 99%+enantiomeric excess (99% ee) pure (+)-(2S,3S)-2-(3-chlorophenyl)-3,5,5-trimethyl-2-morpholinol salt.

2. Enzyme-Catalyzed Dynamic Kinetic Resolution Process to Prepare (+)-(2S,3S)-2-(3-chlorophenyl)-3,5,5-trimethyl-2-morpholinol and Its Pharmaceutically Acceptable Salts

Step One: Dissolving (+/−)-(2R*,3R*)-2-(3-chlorophenyl)-3,5,5-trimethyl-2-morpholinol in a solvent and water and adjusting the pH from about pH 1 to about pH 8.

In this step, (+/−)-(2R*,3R*)-2-(3-chlorophenyl)-3,5,5-trimethyl-2-morpholinol is dissolved in a solvent and water. The amount of water employed is at least about 1% to hydrate the enzyme active site. Typically, the amount of water employed is between about 1% to about 25% based upon the solubility of the racemic (+/−)-(2R*,3R*)-2-(3-chlorophenyl)-3,5,5-trimethyl-2-morpholinol and the insolubility of the desired (+)-(2S,3S)-2-(3-chlorophenyl)-3,5,5-trimethyl-2-morpholinol free base or desired (+)-(2S,3S)-2-(3-chlorophenyl)-3,5,5-trimethyl-2-morpholinol acid salt.

Suitable solvents include protic solvents such as alcohols such as methanol and ethanol; ketones such as acetone; ethers such as methyl t-butyl ether; cyclic ethers such as tetrahydrofuran; nitrites such as acetonitrile; amides such as dimethylformamide; sulfoxides such as dimethyl sulfoxide, and the like including mixtures of solvents. Typical concentrations of the racemate morpholinol in a given solvent or solvent combination are about 0.01 molar to about 2.0 molar. The type and amount of solvent should be selected so as to completely or substantially dissolve the (+/−)-(2R*,3R*)-2-(3-chlorophenyl)-3,5,5-trimethyl-2-morpholinol. Use of the above-described types and amount of solvent allows the enzymatic kinetic resolution to take place efficiently leading to higher yields of the desired chiral end-product: (+)-(2S,3S)-2-(3-chlorophenyl)-3,5,5-trimethyl-2-morpholinol free base and/or acid salts.

The pH is adjusted such that it is within the range of pH from about pH 1 to about pH 8. The dissolved (+/−)-(2R*,3R*)-2-(3-chlorophenyl)-3,5,5-trimethyl-2-morpholinol is treated with an acid, a base, or a combination of acid and base as needed to adjust the pH of the solution to a pH from about pH 1 to about pH 8.

Suitable acids include, but are not limited to, inorganic acids (e.g., hydrochloric acid, sulfuric acid, nitric acid, and phosphoric acid) and organic acids (e.g., acetic acid, benzoic acid, formic acid, and the like).

Suitable bases include, but are not limited to, tertiary amine bases such as trimethylamine and triethylamine; aromatic bases such as pyridine, collidine, and the like; inorganic bases such as ammonium hydroxide, sodium bicarbonate, ammonium carbonate, sodium carbonate and sodium hydroxide. The dissolved racemate to acid ratio would be about 100:1 to about 1:2.

The temperature of the reaction solution can vary from about 10° C. to about 50° C. and will vary depending on the particular esterase enzyme or lipase enzyme that is employed in a given process. A given temperature range is maintained by the application of heat or cooling using means known to those skilled in the chemical arts.

Step Two: Adding a catalytic amount of an esterase enzyme or a lipase enzyme optionally with stirring.

Preferably, the esterase enzyme or lipase enzyme is employed in an amount ranging from about 0.001 grams to about 10 grams of enzyme per gram molecular weight of the racemic substrate. Preferably the esterase enzyme and/or lipase enzyme is added slowly (such as dropwise or in small amounts or aliquots).

The use of esterase enzyme or lipase enzyme is illustrated in the schematic diagram above. Esterase enzyme or lipase enzyme allows the undesired 2R,3R-morpholinol (co-equal structures 3 and 4) to enzymatically “unravel” to the undesired 2R-hydroxybupropion (intermediate D), then to be enzymatically racemized or equilibrated (intermediate D→intermediate C→intermediate B)→intermediate A) with the desired 2S-hydroxybupropion (intermediate A), which then enzymatically “ravels” back up to the desired 2S,3S-morpholinol (co-equal structures 1 and 2) which precipitates or crystallizes out as a solid free base, solid acid salt, or mixture thereof. It is believed that this method will produce a desired free base or solid acid salt in about 80% to about 100% yield, preferably about 90% to about 100% yield, with little or no mother liquor left over. Generally, a chiral acid resolving agent is not needed in this process.

As used herein, an amine-containing compound is commonly termed an amine free base or “free base” if the amine exists in a non-protonated or non-salt form.

Examples of esterase enzymes and lipase enzymes useful in the process of the invention are shown in Tables 1 and 2, respectively.

TABLE 1
EXAMPLES OF ESTERASE ENZYMES USEFUL
IN THE PROCESS OF THE INVENTION

HORSE LIVER ESTERASE [EC3.1.1.1], from horse liver,
(Aldrich Chemical Company or Sigma Chemical Company)
HUMAN LIVER ESTERASE [EC3.1.1.1], from human liver,
(Aldrich Chemical Company or Sigma Chemical Company)
PIG LIVER ESTERASE [EC3.1.1.1], from porcine liver (PLE),
Sigma Chemical Company, E 3019
PIG LIVER ESTERASE ISOENZYME 1[EC3.1.1.1], from porcine
liver (PLE), Biochemika, Sigma Chemical Company, E 3019
RABBIT LIVER ESTERASE [EC3.1.1.1], from rabbit liver,
Sigma Chemical Company, E 9636
ESTERASE [EC3.1.1.1] from Bacillus sp.,
Biochemika (Aldrich Chemical Company or Sigma Chemical Company)
ESTERASE [EC3.1.1.1] from Mucor miehei,
Biochemika (Aldrich Chemical Company or Sigma Chemical Company)
ESTERASE [EC3.1.1.1] from Rhizopus oryzae,
Biochemika (Aldrich Chemical Company or Sigma Chemical Company)
ESTERASE [EC3.1.1.1] from Rhizomucor miehei,
Biochemika (Aldrich Chemical Company or Sigma Chemical Company)
ESTERASE [EC3.1.1.1] from Saccharomyces cerevisiae,
Biochemika (Aldrich Chemical Company or Sigma Chemical Company)
ESTERASE [EC3.1.1.1] from Streptomyces diastalochromgenes,
Biochemika (Aldrich Chemical Company or Sigma Chemical Company)
ESTERASE [EC3.1.1.1] from Stearotherophilus,
Biochemika (Aldrich Chemical Company or Sigma Chemical Company)
ESTERASE [EC3.1.1.1] from Thermoanaerobium brockii,
Biochemika (Aldrich Chemical Company or Sigma Chemical Company)
ESTERASE [EC3.1.1.1] from Thermomyces lanugenosus,
Biochemika (Aldrich Chemical Company or Sigma Chemical Company)

TABLE 2
EXAMPLES OF LIPASE ENZYMES USEFUL
IN THE PROCESS OF THE INVENTION

LIPASE [EC3.1.1.3] from Aspergillus oryzae,
Biochemika (Aldrich Chemical Company, 62285)
LIPASE [EC3 1.1.3] from Candida antarctica,
Biochemika (Aldrich Chemical Company, 62299)
LIPASE [EC3.1.1.3] from Candida cylindracea,
Biochemika (Aldrich Chemical Company, 62302)
LIPASE [EC3.1.1.3] from Candida utilis,
Biochemika (Aldrich Chemical Company, 62307)
LIPASE [EC3.1.1.3] from Mucor javanicus,
Biochemika (Aldrich Chemical Company, 62304)
LIPASE [EC3.1.1.3] from Mucor miehei,
Biochemika (Aldrich Chemical Company, 62298)
LIPASE from Pseudomonas fluorescens,
(Aldrich Chemical Company, 39,044-5)
LIPASE from Pseudomonas fluorescens,
P-30 Amano (Amano Enzyme Company, P-30)
LIPASE from Pseudomonas fluorescens,
P-800 Amano (Amano Enzyme Company, P-800)
LIPASE [EC3.1.1.3] from Rhizomucor arrhizus,
Biochemika (Aldrich Chemical Company, 62305)
LIPASE [EC3.1.1.3] fromRhizomucor miehei,
Biochemika (Aldrich Chemical Company, 62291)
LIPASE [EC3.1.1.3] from Thermus flavus,
Biochemika (Aldrich Chemical Company, 62295)
LIPASE [EC3.1.1.3] from Thermus thermophilus,
Biochemika (Aldrich Chemical Company, 62296)
LIPASE, IMMOBLIIZED In Sol-Gel-AK [EC3.1.1.3] from
Aspergillus niger, Biochemika (Aldrich Chemical Company, 62281)
LIPASE, IMMOBLIIZED in Sol-Gel-AK [EC3.1.1.3] from
Mucor miehei, Biochemika (Aldrich Chemical Company, 62282)
LIPASE, IMMOBLIIZED in Sol-Gel-AK [EC3.1.1.3] from Pseudomonas
fluorescens, Biochemika (Aldrich Chemical Company, 62283)
LIPASE, IMMOBLIIZED in Sol-Gel-AK from Candida
antarctica, Biochemika (Aldrich Chemical Company, 62277)
LIPASE, IMMOBLIIZED in Sol-Gel-AK from Candida
cylindracea, Biochemika (Aldrich Chemical Company, 62278)
LIPASE, IMMOBLIIZED in Sol-Gel-AK from Pseudomonas
cepacia, Biochemika (Aldrich Chemical Company, 62279)
LIPASE B, recombinant [EC3.1.1.3] from Candida
antarctica, Biochemika (Aldrich Chemical Company, 62288)
LIPASE, PORCINE PANCREAS (PPL) from Porcine Pancreas,
Sigma Chemical Company, L 0382
LIPASE, HUMAN PANCREAS (HPL) from Human Pancreas,
Sigma Chemical Company, L 9780

As necessary the pH of the solution is maintained within a pH range of about pH 1 to about pH 8 with acid, base, or combination thereof as previously set forth in Step One above. A pH within this range is desired, and will vary within the disclosed pH range from about pH 1 to about pH 8 depending upon the esterase or lipase chosen for each enzyme-catalyzed dynamic kinetic resolution reaction. Stirring, when employed, is accomplished by means well-known to those skilled in the art. Slow addition of the enzyme to the reaction mixture may be advantageous.

Step Three: Adding seed crystals selected from the group consisting of (i) (+)-(2S,3S)-2-(3-chlorophenyl)-3,5,5-trimethyl-2-morpholinol free base, (ii) a salt of (+)-(2S,3S)-2-(3-chlorophenyl)-3,5,5-trimethyl-2-morpholinol, and (iii) a mixture of said free base and said salt of (+)-(2S,3S)-2-(3-chlorophenyl)-3,5,5-trimethyl-2-morpholinol, while maintaining the pH between about pH 1 and about pH 8 and the reaction temperature between about 10 C.°and about 50° C.

The amount of seed crystals added includes, but is not limited to about 10 mg (for small lab scale reactions of about 10 mL total reaction volume) to about 1.0 gram (for large scale reactions of about a liter to multi-liter total reaction volumes) with stirring at about 10 to about 100 revolutions per minute (rpms).

If the stirring is too fast, the enzyme may degrade due to shearing forces on the enzyme protein. If the stirring is too slow, precipitation or crystallization of the desired product may not be uniform or may occur too quickly leading to a large amount of solid in a ball or clump. This is undesirable and may also cause the stirring shaft to break or the stirring shaft motor to overheat.

The stirring may be provided by any means, for instance, by mechanical stirring with one of a number of types of commercially available paddle stirrers, known to those in the mixing art. Plastic paddles or metal paddles without sharp edges are preferred.

The specific enzyme chosen, the pH, and the temperature chosen optimize each enzyme-catalyzed dynamic kinetic resolution reaction as is known by one skilled in the art. In this step, the pH is maintained between about pH 1 to about pH 8, and varies depending upon the particular enzyme that is employed in a given reaction. Again, the pH is important in stabilizing the esterase or lipase protein enzyme while performing the enzymatically-catalyzed dynamic kinetic resolution.

The employment of esterase or lipase as illustrated in the schematic diagram (above) allows the undesired 2R,3R-morpholinol (co-equal structures 3 and 4) to enzymatically “unravel” to the undesired 2R-hydroxybupropion (intermediate D), then to be enzymatically racemized or equilibrated (intermediate D→intermediate C→intermediate B)→intermediate A) with the desired 2S-hydroxybupropion (intermediate A), which then enzymatically “ravels” back up to the desired 2S,3S-morpholinol (co-equal structures 1 and 2) which precipitates or crystallizes out as a solid free base or solid acid salt or a mixture thereof. This method will produce a desired free base or solid acid salt in about 80 to about 100% yield, preferably about 90 to about 100% yield, with little or no mother liquor left over. Generally, a chiral acid resolving agent is not needed in this process.

If the pH is too high, (a pH of about pH 12 to about pH 14), the esterase or lipase may degrade and denature. At pH of about pH 12 to about pH 14 these conditions may also degrade the (+)-(2S,3S)-2-(3-chlorophenyl)-3,5,5-trimethyl-2-morpholinol free base to a number of by-products, including the degradant—the basic salt of meta-chlorobenzoic acid (i.e. meta-chlorobenzoic acid, sodium salt, as an example).

If the pH is too low, (about pH 1), there may be no equilibration of the (−)-(2R,3R)-2-(3-chlorophenyl)-3,5,5-trimethyl-2-morpholinol to the (+)-(2S,3S)-2-(3-chlorophenyl)-3,5,5-trimethyl-2-morpholinol via the 2R- and 2S-hydroxybupropion molecules. This equilibration step is both pH and enzyme dependent (see schematic diagram above).

Additional acid, base, or both may be added to maintain the desired pH. Any of the above-listed acids or bases are suitable. However, preferably the same acid or base is used throughout the present enzyme-catalyzed dynamic kinetic resolution process.

Step Four: Stopping the enzymatic reaction by quenching the reaction with an organic solvent and a base.

In this step, the solid mixture of esterase enzyme or lipase enzyme and desired reaction product (+)-(2S,3S)-2-(3-chlorophenyl)-3,5,5-trimethyl-2-morpholinol free base or acid salt thereof such as, for example, (+)-(2S,3S)-2-(3-chlorophenyl)-3,5,5-trimethyl-2-morpholinol hydrochloride salt or hydrogen sulfate salt is “partitioned” between an organic phase (solvent) phase and basic aqueous phase (base). By partitioning is meant, the separation of two insoluble or nearly insoluble phases in a two-phased liquid mixture.

In this step, any crystalline acid salts that are present are converted into the (+)-(2S,3S)-2-(3-chlorophenyl)-3,5,5-trimethyl-2-morpholinol free base. This may be accomplished by making the aqueous base solution from a strong base such as ammonium hydroxide, potassium hydroxide or sodium hydroxide in water to pH greater than about pH 10. In this aqueous phase, the pH should be basic enough to convert all of the acids to their basic water-soluble salts (i.e., for example, to an ammonium salt or sodium salt).

The organic phase is made up of an organic solvent, e.g., methylene chloride, ethyl acetate, methyl t-butyl ether, and the like. The type and amount of organic solvent should be selected to completely or substantially extract the (+)-(2S,3S)-2-(3-chlorophenyl)-3,5,5-trimethyl-2-morpholinol free base into the organic phase. The amount of solvent employed is not critical. In general, it should be an amount sufficient to conduct the extraction without an undue amount of waste or “used” solvent so as to minimize the amount of solvent that is disposed or recycled.

Step Five: Removing said esterase enzyme or said lipase enzyme from the reaction.

With the desired free base form of the (+)-(2S,3S)-2-(3-chlorophenyl)-3,5,5-trimethyl-2-morpholinol in the organic phase and the acid salt form of the (+)-(2S,3S)-2-(3-chlorophenyl)-3,5,5-trimethyl-2-morpholinol in the aqueous phase, filtration of both phases together (e.g., through a sintered glass filter funnel, a Gooch filter, a pan filter, or a Rosemund filter) will remove/recover the insoluble esterase or lipase enzyme protein which is insoluble in both the organic and aqueous phases. The removed/recovered esterase or lipase enzyme then may be re-cycled and re-used in future enzymatically controlled dynamic kinetic resolution reactions. The enzyme can be washed with additional organic phase solvent and water and stored as a wet cake at ambient temperature or refrigerated temperatures for future re-use.

Step Six: Isolation of the (+)-(2S,3S)-2-(3-chlorophenyl)-3,5,5-trimethyl-2-morpholinol free base.

The organic phase from Step Five contains (+)-(2S,3S)-2-(3-chlorophenyl)-3,5,5-trimethyl-2-morpholinol free base that is separated from the aqueous phase by phase partitioning means well known in the art. Additional free base can be isolated from the filtered aqueous phase by extraction with the organic solvent (e.g., methylene chloride, ethyl acetate, methyl t-butyl ether, and the like).

The type and amount of organic solvent should be selected so as to completely, or substantially, extract the free base into the organic phase. If not enough organic solvent is used, not all of the free base will be extracted from the aqueous phase into the organic phase. If too much organic solvent is used, the final evaporation of the organic phase will take longer than necessary. The combined organic phases are separated from the aqueous phase via phase partitioning by methods known in the art. Evaporation of the combined organic phase then can yield the desired (+)-(2S,3S)-2-(3-chlorophenyl)-3,5,5-trimethyl-2-morpholinol free base.

Step Seven: Conversion of the (+)-(2S,3S)-2-(3-chlorophenyl)-3,5,5-trimethyl-2-morpholinol free base into (+)-(2S,3S)-2-(3-chlorophenyl)-3,5,5-trimethyl-2-morpholinol salt.

This step may be performed by addition of more than one equivalent of an inorganic acid (e.g., hydrochloric acid, sulfuric acid, phosphoric acid, nitric acid, and the like) or an organic acid (e.g., acetic acid, benzoic acid, formic acid, and the like) and a co-solvent (or, alternatively, by addition of more than one equivalent of acid such as hydrogen chloride gas and a co-solvent), such that the pH of the solution or mixture reaches pH from about pH 1 to about pH 2. Of these acids, hydrochloric acid is preferred. The amount of either acid (e.g., hydrochloric acid) or gas (e.g., hydrogen chloride) should be selected so as to completely, or substantially, convert the free base into the salt (e.g., hydrochloride salt form). Other pharmaceutically acceptable salts (or salt forms) that may be formed can include, but are not limited to, hydrogen chloride salt, hydrogen sulfate salt and other sulfate salts, hydrogen phosphate salt and other phosphate salts, methanesulfonate salt, p-toluenesulfonate salt, citrate salt, fumarate salt, tartrate salt, and the like. Of these, (+)-(2S,3S)-2-(3-chlorophenyl)-3,5,5-trimethyl-2-morpholinol hydrochloride salt is preferred. If not enough of the acid (e.g., the hydrochloric acid or hydrochloride gas) is used, the conversion will be incomplete, and the yield will, therefore, be reduced. If too much of the acid (e.g., the hydrochloric acid or hydrogen chloride) is used, there should be no problem other than excess waste generation.

Also, the type and amount of co-solvent should be selected to aid in the dissolving of the (+)-(2S,3S)-2-(3-chlorophenyl)-3,5,5-trimethyl-2-morpholinol free base and/or to aid in the crystallization or precipitating of the desired final product (e.g., (+)-(2S,3S)-2-(3-chlorophenyl)-3,5,5-trimethyl-2-morpholinol hydrochloride salt). Amounts and ranges of hydrogen chloride or hydrochloric acid and solvents are at least about 1 equivalent of hydrogen chloride in an organic solvent or 1 equivalent of hydrochloric acid (i.e., hydrogen chloride in aqueous solvent). Suitable solvents include methanol, ethanol, ethyl acetate, isopropyl acetate, acetonitrile, and the like. Suitable co-solvents can include ethers such as diethyl ether, methyl tert-butyl ether, diphenyl ether; aromatic hydrocarbons such as benzene or toluene; and/or aliphatic hydrocarbons such as hexane or heptane.

For example, (+)-(2S,3S)-2-(3-chlorophenyl)-3,5,5-trimethyl-2-morpholinol hydrochloride salt is dissolved in methanol, filtered and the co-solvent ethyl acetate is added. Under vacuum, sufficient methanol is removed so that the (+)-(2S,3S)-2-(3-chlorophenyl)-3,5,5-trimethyl-2-morpholinol hydrochloride salt precipitates or crystallizes from a solution comprising mainly ethyl acetate (about 50% to about 100% of the original solvent volume).

The amount of solvent present should be enough to prepare about 0.1 molar to about 4.0 molar solutions. Typically the co-solvent is present in an amount of about 10% to about 100% of the solvent volume.

Step Eight: Recrystallizing the salt of Step Seven to produce a purer form of a pharmaceutically salt such as (+)-(2S,3S)-2-(3-chlorophenyl)-3,5,5-trimethyl-2-morpholinol hydrdrochloride salt.

A final recrystallization is performed by conducting a polishing filtration, followed by precipitation or crystallization with at least one organic solvent. To carry out a “final crystallization” of a drug substance to meet regulatory guidelines and regulations in the great majority of cases, it is necessary to fully dissolve the material to be crystallized, and then filter this solution.

By a “polishing filtration”, is meant the removal of trace amounts of extraneous material (e.g., dust, paper, and cloth fibers that may be present in small amounts), prior to the final crystallization. Accordingly, the amount of solvent present must be sufficient to dissolve all of the (+)-(2S,3S)-2-(3-chlorophenyl)-3,5,5-trimethyl-2-morpholinol hydrochloride salt. If too little solvent is used, it will not be possible to dissolve all of the hydrochloride salt and accomplish this needed recrystallization procedure including the “polishing filtration purification”. Too much solvent will result in lower yields of final crystalline product (+)-(2S,3S)-2-(3-chlorophenyl)-3,5,5-trimethyl-2-morpholinol hydrochloride salt.

Suitable organic solvents include methanol, ethanol, ethyl acetate, isopropyl acetate, acetonitrile, mixtures thereof, and the like. The initial concentration of the (+)-(2S,3S)-2-(3-chlorophenyl)-3,5,5-trimethyl-2-morpholinol hydrochloride salt in the organic solvent or solvent mixture ranges from about 0.1 molar to about 4.0 molar and is capable of being filtered to remove insoluble impurities such as dust and related particulate matter.

A further invention herein is the providing a pharmaceutical composition comprising an active ingredient of (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with the process(es) described hereinabove together with at least one pharmaceutically acceptable excipient.

The pharmaceutical composition may comprise one or more pharmaceutically acceptable carriers, diluents, and/or excipients. The carrier(s), diluent(s), and/or excipients should be acceptable in the sense of being compatible with the other ingredients of the formulation and not deleterious to the recipient thereof.

Pharmaceutical compositions may be presented in unit dose form containing a predetermined amount of active ingredient per unit dose. Such a unit may contain a therapeutically effective dose of the compound, salt or solvate of the compound, or a fraction of a therapeutically effective dose (i.e., a sub-dose), such that multiple unit dosage forms might be administered at a given time to achieve the desired therapeutically effective dose. Preferred unit dosage formulations are those containing a daily dose or sub-dose, or an appropriate fraction thereof of an active ingredient. Such pharmaceutical compositions may be prepared by any of the methods well known in the pharmacy art.

The precise therapeutically effective amount of active ingredient will depend on a number of factors including, but not limited to, the age and weight of the subject being treated, the precise disorder requiring treatment and its severity, the nature of the formulation, and the route of administration, and will ultimately be at the discretion of the attendant physician or veterinarian. Typically, the dose given for treatment will range from about 0.001 mg/kg to about 30 mg/kg body weight of recipient (animal) per day and more usually in the rage of about 0.01 mg/kg to about 20 mg/kg body weight per day. In general, acceptable daily dosages, may be from about 0.1 mg/day to about 3000 mg/day, and preferably from about 0.1 mg/day to about 2000 mg/day. Unit doses will normally be administered once or more than once per day, preferably about 1 to about 4 times per day.

Pharmaceutical compositions may be adapted for administration by any appropriate route, for example, by oral (including buccal or sublingual), rectal, nasal, topical (including buccal, sublingual, or transdermal), vaginal or parenteral (including subcutaneous, intramuscular, intravenous or intradermal) route. Such compositions may be prepared by any method known in the art of pharmacy, for example, by bringing into association the active ingredient with the carrier(s), diluent(s), and/or excipient(s). Oral administration is most preferred.

One or more compounds prepared by the inventive process may be present with one or more non-toxic pharmaceutically acceptable ingredients and optionally, other active anti-proliferative agents, to form the pharmaceutical composition. These compositions can be prepared by applying known techniques in the art such as those taught in Remington's Pharmaceutical Sciences (Fourteenth Edition), Managing Editor, John E. Hoover, Mack Publishing Co., (1970) or Pharmaceutical Dosage Form and Drug Delivery Systems (Sixth Edition), edited by Ansel et al., publ. by Williams & Wilkins, (1995).

Depending on the route of administration, the composition can take the form of discrete units such as aerosols, creams, elixirs, emulsions, foams, whips, gels, granules, wafers, candy, inhalants, lotions, magmas, ointments, peroral solids, quick-dissolve tongue tapes (or sheets), powders, sprays, syrups, suppositories, suspensions, tablets, capsules, and tinctures. Tablets, capsules, granules, and powders are preferred. Tablets and capsules are most preferred. A once-daily tablet is most preferred. Ways of preparing these discrete units are well known in the formulation arts.

In still another embodiment of the invention there is provided a method of treatment comprising the administration (preferably oral) to a mammal of a therapeutically effective amount of an active ingredient comprising (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with the process described herein together with at least one pharmaceutically acceptable excipient. As used herein, the term “treatment” refers to alleviating the specified condition, eliminating or reducing one or more symptoms of the condition, slowing or eliminating the progression of the condition, and preventing or delaying the reoccurrence of the condition in a previously afflicted or diagnosed patient or subject. As used herein, the term “therapeutically effective amount” means an amount of the active ingredient which is sufficient, in the subject to which it is administered, to elicit the biological or medical response of a cell culture, tissue, system, animal (including human), that is being sought for instance by a physician, researcher, or clinician.

In another embodiment there is provided the use of (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with process described herein in the manufacture of a medicament. Such medicament can be used for the treatment of depression (major and bipolar), attention deficit hyperactivity disorder (ADHD), anxiety, obesity, migraine, pain, sexual dysfunction in both men and women, Parkinson's disease, Alzheimer's disease, seasonal affective disorder (SAD), addiction to alcohol, addiction to cocaine, or addiction to nicotine-containing products (e.g., tobacco).

### EXAMPLES

### Experiment 01a

### Preparation of Chiral Free Base Using a Lipase Enzyme

A total of 255.8 gm (1.0 mole) of (+/−)-(2R*,3R*)-2-(3-chlorophenyl)-3,5,5-trimethyl-2-morpholinol is dissolved in 2.0 liters of acetone containing about 1% water and stirred at room temperature and enough 0.1 N hydrochloric acid is added to bring the pH of this solution to about pH 6 to about pH 7.

As this solution is slowly mechanically stirred, portions totaling 5.0 gm of lipase from Pseudomonas fluorescens, P-30 Amano (Amano Enzyme Company, P-30) is added along with 0.1 N hydrochloric acid and 0.1 N sodium hydroxide to maintain this now heterogeneous mixture at pH 6 to pH 7. At this point the heterogenous solution is seeded with 0.5 gm each of (+)-(2S,3S)-2-(3-chlorophenyl)-3,5,5-trimethyl-2-morpholinol free base and its hydrochloride salt. The mixture is stirred for 16 hours to aid the precipitation of desired solid forms of (+)-(2S,3S-2-(3-chlorophenyl)-3,5,5-trimethyl-2-morpholinol free base and its hydrochloride salt from the solution.

The reaction mixture containing (+)-(2S,3S)-2-(3-chlorophenyl)-3,5,5-trimethyl-2-morpholinol free base and its hydrochloride salt is partitioned into an organic phase and an aqueous phase after stirring with a 1:1 mixture of 2.5 liters of methylene chloride and 2.5 liters of de-ionized water containing 3.0-3.5 equivalents of aqueous ammonium hydroxide base (a pH greater than 10). The two phases containing insoluble lipase protein at the interface are filtered to remove the lipase enzyme protein. An additional 1:1 mixture of 500 ml of methylene chloride and 500 ml of de-ionized water are stirred with the collected lipase enzyme protein and filtered. The insoluble filter cake of recovered lipase enzyme protein is bottled and stored in a refrigerator at 4-10 C.° for future re-use.

The organic phase of the combined filtrates is separated from the aqueous phase of the combined filtrates. The separated aqueous phase is extracted with an additional 1.0 liter of methylene chloride. This organic phase resulting therefrom is combined with the previous organic phase. The aqueous phase is discarded. The combined organic phases are washed with 1 liter of de-ionized water. The washed organic phase is separated from the aqueous wash and concentrated under vacuum to yield crude (+)-(2S,3S)-2-(3-chlorophenyl)-3,5,5-trimethyl-2-morpholinol free base.

### Experiment 01b

### Preparation of Chiral Hydrochloride Salt

A total of 200 gm of crude (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a white precipitate forms and the pH of the solution reaches and is maintained at about pH 1 to about pH 2. The mixture is stirred for one hour and cooled to 10° C. for one additional hour with stirring. The desired (+>(2S,3S)-2-(3-chlorophenyl)-3,5,5-trimethyl-2-morpholinol hydrochloride salt is isolated by filtration, washed with 49:1 ethyl acetate-methanol and dried under vacuum. In this manner up to 97% enantiomeric excess (97% ee) pure quality (+)-(2S,3S)-2-(3-chlorophenyl)-3,5,5-trimethyl-2-morpholinol hydrochloride is prepared.

### Experiment 01c

### Polishing Filtration and Crystallization of Chiral Hydrochloride Salt

The (+)-(2S,3S)-2-(3-chlorophenyl)-3,5,5-trimethyl-2-morpholinol hydrochloride is subjected to a polishing filtration and crystallization. This salt, from Experiment 1b, is dissolved in sufficient methanol to give a homogeneous solution which is filtered to remove any inert particulate matter using a sintered glass filter funnel. The filtered solution is diluted with 1-3 volumes of ethyl acetate and concentrated under reduced pressure to selectively remove some of the methanol and to induce crystallization. This now heterogeneous solution is stirred for 2-4 hours at ambient temperature to complete the crystallization process. The solid mass of desired product is collected by filtration and dried. In this manner, 99+% enantiomeric excess (99+% ee) pure quality (+)-(2S,3S)-2-(3-chlorophenyl)-3,5,5-trimethyl-2-morpholinol hydrochloride is prepared.

### Experiment 02a

### Preparation of Chiral Free Base Using a Lipase Enzyme

A total of 255.8 gm (1.0 mole) of (+/−)-(2R*,3R*)-2-(3-chlorophenyl)-3,5,5-trimethyl-2-morpholinol is dissolved in 2.0 liters of acetone containing about 1% water and stirred at room temperature and enough 0.1 N hydrochloric acid is added to bring the pH of this solution to about pH 6 to about pH 7.

As this solution is slowly mechanically stirred, portions totaling 2.0 gm of lipase from Pseudomonas fluorescens, P-800 Amano (Amano Enzyme Company, P-800) was added along with 0.1 N hydrochloric acid and 0.1 N sodium hydroxide to maintain this now heterogeneous mixture at pH 6 to pH 7. At this point the heterogeneous solution is seeded with 0.5 gm each of (+)-(2S,3S)-2-(3-chlorophenyl)-3,5,5-trimethyl-2-morpholinol free base and its hydrochloride salt. The mixture is stirred for 16 hours to aid the precipitation of desired solid forms of (+)-(2S,3S)-2-(3-chlorophenyl)-3,5,5-trimethyl-2-morpholinol free base and its hydrochloride salt from the solution.

The reaction mixture containing (+)-(2S,3S)-2-(3-chlorophenyl)-3,5,5-trimethyl-2-morpholinol free base and its hydrochloride salt is partitioned into an organic phase and an aqueous phase after stirring with a 1:1 mixture of 2.5 liters of methylene chloride and 2.5 liters of de-ionized water containing 3.0-3.5 equivalents of aqueous ammonium hydroxide base (pH greater than 10). The two phases containing insoluble lipase protein at the interface are filtered to remove the lipase enzyme protein. An additional 1:1 mixture of 500 ml of methylene chloride and 500 ml of de-ionized water are stirred with the collected lipase enzyme protein and filtered. The insoluble filter cake of recovered lipase enzyme protein is bottled and stored in a refrigerator at 4-10 C.° for future re-use.

The organic phase of the combined filtrates is separated from the aqueous phase of the combined filtrates. The separated aqueous phase is extracted with an additional 1.0 liter of methylene chloride. This organic phase resulting therefrom is combined with the previous organic phase. The aqueous phase is discarded. The combined organic phases are washed with 1 liter of de-ionized water. The washed organic phase is separated from the aqueous wash and concentrated under vacuum to yield crude (+)-(2S,3S)-2-(3-chlorophenyl)-3,5,5-trimethyl-2-morpholinol free base.

### Experiment 02b

### Preparation of Chiral Hydrochloride Salt

A total of 200 gm of crude (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a white precipitate forms and the pH of the solution reaches and is maintained at about pH 1 to about pH 2. The mixture is stirred for one hour and cooled to 10° C. for one additional hour with stirring. The desired (+)-(2S,3S)-2-(3-chlorophenyl)-3,5,5-trimethyl-2-morpholinol hydrochloride salt is isolated by filtration, washed with 49:1 ethyl acetate-methanol and dried under vacuum. In this manner up to 98% enantiomeric excess (98% ee) pure quality (+)-(2S,3S)-2-(3-chlorophenyl)-3,5,5-trimethyl-2-morpholinol hydrochloride is prepared.

### Experiment 02c

### Polishing Filtration and Crystallization of Chiral Hydrochloride Salt

The (+)-(2S,3S)-2-(3-chlorophenyl)-3,5,5-trimethyl-2-morpholinol hydrochloride is subjected to a polishing filtration and crystallization. This salt, from Experiment 2b, is dissolved in sufficient methanol to give a homogeneous solution which is filtered to remove any inert particulate matter using a sintered glass filter funnel. The filtered solution is diluted with 1-3 volumes of ethyl acetate and concentrated under reduced pressure to selectively remove some of the methanol and to induce crystallization. This now heterogeneous solution is stirred for 2-4 hours at ambient temperature to complete the crystallization process. The solid mass of desired product is collected by filtration and dried. In this manner 99+% enantiomeric excess (99+% ee) pure quality (+)-(2S,3S)-2-(3-chlorophenyl)-3,5,5-trimethyl-2-morpholinol hydrochloride is prepared.

### Experiment 03a

### Preparation of Chiral Free Base Using an Esterase Enzyme

A total of 255.8 gm (1.0 mole) of (+/−)-(2R*,3R*)-2-(3-chlorophenyl)-3,5,5-trimethyl-2-morpholinol is dissolved in 2.0 liters of acetone containing about 1% water and stirred at room temperature and enough 0.1 N sulfuric acid is added to bring the pH of this solution to a pH of about pH 5 to about pH 6.

As this solution is slowly mechanically stirred, portions of esterase [EC3.1.1.1] from Thermoanaerobium brockii , Biochemika (Aldrich Chemical Company) is added along with 0.1 N sulfuric acid and 0.1 N sodium hydroxide to maintain this now heterogeneous mixture at about pH 5 to pH 6. At this point, the heterogeneous solution is seeded with 0.5 gm each of (+)-(2S,3S)-2-(3-chlorophenyl)-3,5,5-trimethyl-2-morpholinol free base and hydrogen sulfate salt. The mixture is stirred for about 16 hours to aid the precipitation of desired solid forms of (+)-(2S,3S)-2-(3-chlorophenyl)-3,5,5-trimethyl-2-morpholinol free base and hydrogen sulfate salt from the solution.

The reaction mixture containing (+)-(2S,3S)-2-(3-chlorophenyl)-3,5,5-trimethyl-2-morpholinol free base and hydrogen sulfate salt is partitioned into an organic phase and an aqueous phase after stirring with a 1:1 mixture of 2.5 liters of methylene chloride and 2.5 liters of de-ionized water containing 3.0-3.5 equivalents of aqueous ammonium hydroxide base (pH greater than 10). The two phases containing insoluble esterase protein at the interface are filtered to remove the esterase enzyme protein. An additional 1:1 mixture of 500 ml of methylene chloride and 500 ml of de-ionized water are stirred with the collected esterase enzyme protein and filtered. The insoluble filter cake of recovered esterase enzyme protein is bottled and stored in a refrigerator at 4-10 C.° for future re-use.

The organic phase of the combined filtrates is separated from the aqueous phase of the combined filtrates. The separated aqueous phase is extracted with an additional 1.0 liter of methylene chloride. This organic phase resulting therefrom is combined with the previous organic phase. The aqueous phase is discarded. The combined organic phases are washed with 1 liter of de-ionized water. This washed organic phase is separated from the aqueous wash and concentrated under vacuum to yield crude (+)-(2S,3S)-2-(3-chlorophenyl)-3,5,5-trimethyl-2-morpholinol free base.

### Experiment 03b

### Preparation of Chiral Hydrochloride Salt

A total of 200 gm of crude (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a precipitate (preferably a white precipitate) forms and the pH of the solution reaches and is maintained at about pH 1 to about pH 2. The mixture is stirred for one hour and cooled to 10° C. for one additional hour with stirring. The desired (+)-(2S,3S)-2-(3-chlorophenyl)-3,5,5-trimethyl-2-morpholinol hydrochloride salt is isolated by filtration, washed with 49:1 ethyl acetate-methanol and dried under vacuum. In this manner up to 97% enantiomeric excess (97% ee) pure quality (+)-(2S,3S)-2-(3-chlorophenyl)-3,5,5-trimethyl-2-morpholinol hydrochloride is prepared.

### Experiment 03c

### Polishing Filtration and Crystallization of Chiral Hydrochloride Salt

If required, the (+)-(2S,3S)-2-(3-chlorophenyl)-3,5,5-trimethyl-2-morpholinol hydrochloride is subjected to a polishing filtration and crystallization. This salt, from Experiment 3b, is dissolved in sufficient methanol to give a homogeneous solution which is filtered to remove any inert particulate matter using a sintered glass filter funnel. The filtered solution is diluted with 1-3 volumes of ethyl acetate and concentrated under reduced pressure to selectively remove some of the methanol and to induce crystallization. This now heterogeneous solution is stirred for 2-4 hours at ambient temperature to complete the crystallization process. The solid mass of desired product is collected by filtration and dried. In this manner 99+% enantiomeric excess (99+% ee) pure quality (+)-(2S,3S)-2-(3-chlorophenyl)-3,5,5-trimethyl-2-morpholinol hydrochloride is prepared.

### Experiment 04a

### Preparation of Chiral Free Base Using an Esterase Enzyme

A total of 255.8 gm (1.0 mole) of (+/−)-(2R*,3R*)-2-(3-chlorophenyl)-3,5,5-trimethyl-2-morpholinol is dissolved in 2.0 liters of acetone containing about 1% water and stirred at room temperature as enough 0.1 N sulfuric acid is added to bring the pH of this solution to about pH 5 to about pH 6.

As this solution is slowly mechanically stirred, portions of esterase [EC3.1.1.1] from Thermomyces lanugenosus, Biochemika (Aldrich Chemical Company) was added along with 0.1 N sulfuric acid and 0.1 N sodium hydroxide to maintain this now heterogeneous mixture at pH 5 to pH 6. At this point, the heterogeneous solution is seeded with 0.5 gm each of (+)-(2S,3S)-2-(3-chlorophenyl)-3,5,5-trimethyl-2-morpholinol free base and hydrogen sulfate salt. The mixture is stirred for 16 hours to aid the precipitation of desired solid forms of (+)-(2S,3S)-2-(3-chlorophenyl)-3,5,5-trimethyl-2-morpholinol free base and hydrogen sulfate salt from the solution.

The reaction mixture containing (+)-(2S,3S)-2-(3-chlorophenyl)-3,5,5-trimethyl-2-morpholinol free base and hydrogen sulfate salt is partitioned into an organic phase and an aqueous phase after stirring with a 1:1 mixture of 2.5 liters of methylene chloride and 2.5 liters of de-ionized water containing 3.0-3.5 equivalents of aqueous ammonium hydroxide base (pH greater than 10). The two phases containing insoluble esterase enzyme protein at the interface are filtered to remove the esterase enzyme protein. An additional 1:1 mixture of 500 ml of methylene chloride and 500 ml of de-ionized water are stirred with the collected esterase enzyme protein and filtered. The insoluble filter cake of recovered esterase enzyme protein is bottled and stored in a refrigerator at 4-10 C.° for future re-use.

The organic phase of the combined filtrates is separated from the aqueous phase of the combined filtrates. The separated aqueous phase is extracted with an additional 1.0 liter of methylene chloride. This organic phase resulting therefrom is combined with the previous organic phase. The aqueous phase is discarded. The combined organic phases are washed with 1 liter of de-ionized water. The washed organic phase is separated from the aqueous wash and concentrated under vacuum to yield crude (+)-(2S,3S)-2-(3-chlorophenyl)-3,5,5-trimethyl-2-morpholinol free base.

### Experiment 04b

### Preparation of Chiral Hydrochloride Salt

A total of 200 gm of crude (+)-(2S,3S)-2-(3-chlorophenyl)-3,5,5-trimethyl-2-morpholinol free base is dissolved in 1 liter of ethyl acetate with heating as necessary. A 5-6 molar solution of hydrochloric acid in methanol is added until a white precipitate forms and the pH of the solution reaches and is maintained at about pH 1 to about pH 2. The mixture is stirred for one hour and cooled to 10° C. for one additional hour with stirring. The desired (+)-(2S,3S)-2-(3-chlorophenyl)-3,5,5-trimethyl-2-morpholinol hydrochloride salt is isolated by filtration, washed with 49:1 ethyl acetate-methanol and dried under vacuum. In this manner up to 97% enantiomeric excess (97% ee) pure quality (+)-(2S,3S)-2-(3-chlorophenyl)-3,5,5-trimethyl-2-morpholinol hydrochloride is prepared.

### Experiment 04c

### Polishing Filtration and Crystallization of Chiral Hydrochloride Salt

If required, the (+)-(2S,3S)-2-(3-chlorophenyl)-3,5,5-trimethyl-2-morpholinol hydrochloride is subjected to a polishing filtration and crystallization. This salt, from Experiment 4b, is dissolved in sufficient methanol to give a homogeneous solution which is filtered to remove any inert particulate matter using either a sintered glass filter funnel. The filtered solution is diluted with 1-3 volumes of ethyl acetate and concentrated under reduced pressure to selectively remove some of the methanol and to induce crystallization. This now heterogeneous solution is stirred for 2-4 hours at ambient temperature to complete the crystallization process. The solid mass of desired product is collected by filtration and dried. In this manner 99+% enantiomeric excess (99+% ee) pure quality (+)-(2S,3S)-2-(3-chlorophenyl)-3,5,5-trimethyl-2-morpholinol hydrochloride is prepared.

All cited patents, publications, co-pending applications, and provisional applications referred to in this application are herein incorporated by reference.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for preparing (+)-(2S,3S)-2-(3-chlorophenyl)-3,5,5-trimethyl-2-morpholinol in its free base, its salt, or both, which process comprises an enzyme-catalyzed dynamic kinetic resolution by equilibrating the two chiral centers of (+/−)-(2R*,3R*)-2-(3-chlorophenyl)-3,5,5-trimethyl-2-morpholinol.

2. The process according to claim 1 which process comprises:
(1) dissolving (+/−)-(2R*,3R*)-2-(3-chlorophenyl)-3,5,5-trimethyl-2-morpholinol in a solvent and water and adjusting the pH from about pH 1 to about pH 8;
(2) adding a catalytic amount of an esterase enzyme or a lipase enzyme optionally with stirring and optionally adjusting the pH from about pH 1 to about pH 8;
(3) adding seed crystals selected from the group consisting of (i) (+)-(2S,3S)-2-(3-chlorophenyl)-3,5,5-trimethyl-2-morpholinol free base, (ii) a salt of (+)-(2S,3S)-2-(3-chlorophenyl)-3,5,5-trimethyl-2-morpholinol, and (iii) a mixture of said free base and said salt of (+)-(2S,3S)-2-(3-chlorophenyl)-3,5,5-trimethyl-2-morpholinol, while maintaining the pH between about pH 1 and about pH 8 and the reaction temperature between about 10 C.° and about 50° C.;
(4) quenching the reaction with an organic solvent and a base;
(5) removing said esterase enzyme or said lipase enzyme from the reaction; and
(6) isolating (+)-(2S,3S)-2-(3-chlorophenyl)-3,5,5-trimethyl-2-morpholinol free base.

3. The process according to claim 2 further comprising the steps of:
(7) converting the (+)-(2S,3S)-2-(3-chlorophenyl)-3,5,5-trimethyl-2-morpholinol free base into a (+)-(2S,3S)-2-(3-chlorophenyl)-3,5,5-trimethyl-2-morpholinol salt; and
(8) recrystallizing said salt to produce a purer form of the (+)-(2S,3S)-2-(3-chlorophenyl)-3,5,5-trimethyl-2-morpholinol salt.

4. The process according to claim 3 wherein the salt is (+)-(2S,3S)-2-(3-chlorophenyl)-3,5,5-trimethyl-2-morpholinol hydrochloride salt.

5. The process according to claim 2 wherein the enzyme is an esterase enzyme.

6. The process according to claim 2 wherein the enzyme is a lipase enzyme.

7. The process according to claim 5 wherein the esterase enzyme is esterase from Thermoanaerobium brockii or esterase from Thermomyces lanugenosus.

8. The process according to claim 6 wherein the lipase enzyme is lipase from Pseudomonas fluorescens.

9. The process according to claim 2 wherein said pH from about pH 1 to about pH 8 is adjusted using (i) an acid, (ii) a base, or (iii) a combination of an acid and a base.

10. The process according to claim 9 wherein said acid used to adjust pH is an organic acid or an inorganic acid; and said base used to adjust pH is an organic base or an inorganic base.

11. The process according to claim 10 wherein said acid used to adjust pH is selected from the group consisting of hydrochloric acid, sulfuric acid, phosphoric acid, and acetic acid.

12. The process according to claim 10 wherein said base used to adjust pH is selected from the group consisting of an alkali metal hydrogen carbonate, an alkali metal carbonate, an alkali metal hydroxide, ammonium hydroxide, an aliphatic amine, and an aromatic amine.

13. The process according to claim 12 wherein the alkali metal hydrogen carbonate is selected from the group consisting of sodium hydrogen carbonate and potassium hydrogen carbonate; the alkali metal carbonate is selected from the group consisting of sodium carbonate and potassium carbonate; and the alkali metal hydroxide is selected from the group consisting of sodium hydroxide and potassium hydroxide.

14. The process according to claim 2 wherein the solvent is selected from the group consisting of a protic solvent, a ketonic solvent, and an ether solvent.

15. The process according to claim 14 wherein said solvent is selected from the group consisting methanol, ethanol, acetone, and tetrahydrofuran.

16. The process according to claim 2 wherein the seed crystal is (i) a (+)-(2S,3S)-2-(3-chlorophenyl)-3,5,5-trimethyl-2-morpholinol hydrochloride salt or (ii) a (+)-(2S,3S)-2-(3-chlorophenyl)-3,5,5-trimethyl-2-morpholinol hydrogen sulfate salt.

17. The process according to claim 2 wherein step (4) has a pH greater than about pH 10.

18. The process according to claim 2 further comprising the step of:
recycling the esterase enzyme or lipase enzyme removed in step (5) into step (2).

19. The process according to claim 2 further comprising the step of:
isolating (+)-(2S,3S)-2-(3-chlorophenyl)-3,5,5-trimethyl-2-morpholinol free base in step (5) such that it is substantially free from enzyme protein in step (5).

20. The process according to claim 19 wherein said isolating is performed using a strong base in an excess amount.

21. The process according to claim 20 wherein said strong base is selected from the group consisting of ammonium hydroxide, potassium hydroxide, and sodium hydroxide in water.

22. The process according to claim 4 wherein said free base is converted into said hydrochloride salt by (i) addition of more than one equivalent of hydrochloric acid and a co-solvent or by (ii) addition of more than one equivalent of hydrogen chloride gas and a co-solvent, such that the pH is between about pH 1 to about pH 2.

23. The process according to claim 22 wherein said co-solvent is at least one selected from the group consisting of methanol, ethanol, ethyl acetate, isopropyl acetate, and acetonitrile.

24. The process according to claim 23 further comprising the step of:
recrystallizing the (+)-(2S,3S)-2-(3-chlorophenyl)-3,5,5-trimethyl-2-morpholinol hydrochloride salt.

25. The process according to claim 24 wherein said recrystallizing is performed by polishing filtration and crystallization in the presence of at least one organic solvent.

26. The process according to claim 25 wherein said organic solvent is selected from the group consisting of methanol, ethanol, ethyl acetate, isopropyl acetate, acetonitrile, and mixtures thereof.

27. The process according to claim 2 wherein said (+)-(2S,3S)-2-(3-chlorophenyl)-3,5,5-trimethyl-2-morpholinol salt is selected from the group consisting a hydrochloride salt, a hydrogen sulfate, a sulfate salt, a hydrogen phosphate, a phosphate salt, a methanesulfonate salt, p-toluenesulfonate salt, a citrate salt, a fumarate salt, and a tartrate salt.

28. The process according to claim 3 wherein said (+)-(2S,3S)-2-(3-chlorophenyl)-3,5,5-trimethyl-2-morpholinol salt is selected from the group consisting a hydrochloride salt, a hydrogen sulfate, a sulfate salt, a hydrogen phosphate, a phosphate salt, a methanesulfonate salt, p-toluenesulfonate salt, a citrate salt, a fumarate salt, and a tartrate salt.

29. A pharmaceutical composition comprising an active ingredient of (+)-(2S,3S)-2-(3-chlorophenyl)-3,5,5-trimethyl-2-morpholinol, a pharmaceutically acceptable salt thereof, or a pharmaceutically acceptable solvate thereof prepared in accordance with claim 1 together with at least one pharmaceutically acceptable excipient.

30. A method of treatment of depression, attention deficit hyperactivity disorder, anxiety, obesity, migraine, pain, sexual dysfunction in both men and women, Parkinson's disease, Alzheimer's disease, seasonal affective disorder, addiction to alcohol, addiction to cocaine, or addiction to nicotine-containing products comprising the oral administration to a mammal of an active ingredient comprising (+)-(2S,3S)-2-(3-chlorophenyl)-3,5,5-trimethyl-2-morpholinol, a pharmaceutically acceptable salt thereof, or a pharmaceutically acceptable solvate thereof, prepared in accordance with claim 1 together with at least one pharmaceutically acceptable excipient.

31. (canceled)
